(19) United States
(12) Patent Application Publication
(10) Pub. No.: US 2007/0122364 A1
(43) Pub. Date: May 31, 2007

(54) Self-tanning facial product having anti-wrinkle/anti-aging properties

(76) Inventors: Tatiana Kelly, Bedford, TX (US); Mary E. Cooper, Oklahoma City, OK (US)

Correspondence Address: DUNLAP, CODDING & ROGERS P.C., PO BOX 16370, OKLAHOMA CITY, OK, 73113, US

(21) Appl. No.: 11/599,787

(22) Filed: Nov. 15, 2006

Related U.S. Application Data
(60) Provisional application No. 60/738,288, filed on Nov. 18, 2005.

(51) Int. Cl. A61K 8/36

(52) U.S. Cl. 424/59

(57) ABSTRACT

A sunless facial self-tanning composition having anti-wrinkle/anti-aging properties. The composition contains safe effective amounts of sunless or self-tanning agents and peptides having anti-wrinkle/anti-aging properties. A method for preparing such composition is disclosed.

### CROSS REFERENCE TO RELATED APPLICATIONS

This application claims benefit under 35 U.S.C. 119(e) of U.S. Ser. No. 60/738,288, filed Nov. 18, 2005.

### FIELD OF INVENTION

The present invention relates generally to self-tanning compositions, and more particularly to self-tanning facial compositions containing anti-wrinkle agents.

### BACKGROUND OF THE INVENTION

Many people use self-tanning compositions to achieve the look of a suntan without exposure to the sun, to obtain a darker suntan or to extend the natural life of a suntan. Most self-tanning compositions affect only the stratum corneum or outermost layer of the epidermis. In addition to or alternatively, many people wish to apply a self-tanning composition to the facial and neck areas to complement the results of an application of a self-tanner to the body.

Tans produced by the prior art sunless or self-tanning compositions have encountered several problems. For example, tans produced by the prior art sunless or self-tanning compositions generally fade within 3 to 5 days due to the sloughing off or wearing away of dead skin cells from the surface of the skin. For this reason, it is generally desirable to reapply the sunless or self-tanning composition frequently to maintain the tan appearance. In addition, problems may arise in using sunless or self-tanning compositions because of the difficulty in obtaining uniform application of the sunless or self-tanning compositions, and the time required for such compositions to dry.

Further, many people desire to use anti-wrinkle or anti-aging formulations on the face and neck area for prevention and/or reduction in the amount of wrinkles on the face and neck. Numerous commercial compositions are available as anti-wrinkle or anti-aging formulations to reduce deep lines or wrinkles in the forehead or around the eyes or on the neck of a person.

However, prior to the present invention, self-tanning composition formulated specifically for the facial and neck area have not incorporated anti-wrinkle or anti-aging agents or formulation to provide a sunless or self-tanning composition having anti-wrinkle and/or anti-aging properties associated therewith. Therefore, a need exists for self-tanning composition containing anti-wrinkle or anti-aging properties which are especially formulated for the facial or neck. It is to such facial sunless and self-tanning compositions having anti-wrinkle and anti-aging properties associated therewith, and methods for producing such facial sunless or self-tanning compositions that the present invention is directed.

### SUMMARY OF THE INVENTION

Broadly, the present invention relates to emulsified facial sunless or self-tanning compositions formulated from a water phase and an oil phase having anti-wrinkle and/or anti-aging properties associated therewith. The water phase contains a major amount of water and safe and effective minor amounts of viscosity modifiers, colorants, at least one chelating agent and preservatives. The oil phase contains safe and effective amounts of peptides which functions as anti-wrinkle/anti-aging agents, self-tanning agents, skin conditioners, and safe and effective minor amounts of emollients, viscosity modifiers, opacifiers, moisturizers, preservatives and fragrances. In addition, the composition contains effective amounts of pH adjusters to provide the composition with a pH in the range of from about 3.25 to about 3.75.

The present invention also relates to a method of producing emulsified facial sunless or self-tanning compositions having anti-wrinkle/anti-aging properties.

### DETAILED DESCRIPTION

As previously stated, the present invention relates to a facial sunless or self-tanning composition having anti-wrinkle and/or anti-aging properties associated therewith. The composition is formulated of a water phase and an oil phase. The water phase contains a major amount of water and safe and effective minor amounts of a viscosity modifier, colorants, at least one chelating agent and preservatives. The oil phase, which is admixed with the water phase in a plurality of stages, contains safe and effective amounts of sunless or self-tanning agents to provide the composition with the desired self-tanning properties, and safe and effective amount of anti-wrinkle/anti-aging agents to provide the resulting composition with the desired anti-wrinkle/anti-aging properties, and safe and effective amounts of emollients, viscosity modifiers, opacifers, film formers, preservatives, solubilizers, skin conditioners, moisturizers and fragrances. In addition, the oil phase is provided with an effective amount of pH adjusters so that the resulting composition has a pH in the range of from about 3.25 to about 3.75.

While the amount of each constituent employed in the formulation of the water phase of the composition of the present invention can vary widely, generally the water phase contains, in addition to water, from about 0.1 to about 0.5 weight percent of a viscosity modifier, from about 0.003 to about 0.9 weight percent of a colorant, from about 0.01 to about 0.5 weight percent of a chelating agent and from about 0.05 to about 0.5 weight percent of a preservative.

Similarly, the amount of each constituent employed in the formulation of the various phases of the oil phase of the composition of the present invention can also vary widely. Generally, however, the oil phase of the composition (i.e. the combination of the plurality of phases which make up the oil phase of the composition) contains from about 0.5 to about 17 weight percent sunless or self-tanning agents, from about 0.1 to about 10 weight percent of anti-wrinkle/anti-aging agents which are compatible with the sunless or self-tanning agents, from about 0.01 to about 15 weight percent skin conditioners, from about 0.1 to about 6 weight percent moisturizers, from about 1.5 to about 14 weight percent emollients and from about 0.5 to about 4 weight percent film formers. In addition, the composition further includes effective minor effective amounts of pH adjusters which provide the composition with a desired pH in the range of about 3.25 to about 3.75 and safe and effective minor amounts of opacifiers, at least one colorant to provide the composition with a predetermined color, chelating agents and fragrances to provide the composition with a pleasing aroma. It is important that each of the ingredients be employed in a safe and effective amount so as to provide the composition with the desired effects of such constituents, namely, sunless or self-tanning properties and anti-wrinkle/anti-aging properties.

As will be described in more detail hereinafter, the facial sunless or self-tanning compositions of the present inventions having anti-wrinkle/anti-aging properties associated therewith are emulsified compositions formulating by admixing various phases of the oil phase with a water phase. The constituents of the water phase and each of the various phases of the oil phase are as follows:

### EXAMPLE

| | | Applicable | |
|---|---|---|---|
| Phase | Ingredient | % w/w⁽¹⁾ | Function |
| A | Deionized Water | 30.00-85.00 | Base |
| | Xanthan Gum | 0.01-0.50 | Thickner or viscosity |
| | | | modifier |
| | D&C Green No. 5 | 0.0001-0.30 | Colorant |
| | FD&C Red No. 40 | 0.0001-0.30 | Colorant |
| | FD&C Yellow No. 5 | 0.0001-0.30 | Colorant |
| | Tetrasodium EDTA | 0.01-0.50 | Chelating Agent |
| | Methylparaben | 0.05-0.50 | Preservative |
| B | Simmondisia | 0.50-5.00 | Emollient |
| | Chinensis (Jojoba) | | |
| | Oil | | |
| | Ethylhexyl Stearate | 0.50-5.00 | Emollient |
| | Dimethicone | 0.50-4.00 | Emollient |
| | Cetyl Esters | 0.10-4.00 | Thickner |
| | Cyclophentasiloxane | 0.10-5.00 | Emollient |
| | Cetearyl Alcohol | 0.25-3.00 | Opacifier |
| | Ceteareth-20 | 0.25-3.00 | Emulsifier |
| | Trimethylpentanediol/ | 0.50-4.00 | Film Former |
| | Adipic Acid | | |
| | Copolymer | | |
| | PEG-100 Stearate | 0.50-4.00 | Emulsifier |
| | Glyceryl Stearate | 0.50-4.00 | Emulsifier |
| | Propylparaben | 0.05-0.50 | Preservative |
| C | Sepigel 305 | 0.25-5.00 | Emulsifier |
| | (Polyacrylamide, | | |
| | C13-14 Isoparaffin, | | |
| | Laureth-7) | | |
| D | Lactic Acid | 0.10-8.00 | pH Adjuster |
| | Triethanolamine | 0.01-1.00 | pH Adjuster |
| E | Deionized Water | 2.0-8.00 | Base |
| | Ethoxydiglycol | 0.10-8.00 | Solubilizer |
| | Dihydroxyacetone | 0.50-12.00 | Self-Tanning |
| F | Matrixyl ™ 3000 | 0.01-10.00 | Peptide in |
| | (Glycerin, Butylene | | Hydroglycolic solution |
| | Glycol, Carbomer, | | |
| | Polysorbate-20, | | |
| | Palmitoyl | | |
| | Oligopeptide, | | |
| | Palmitoyl | | |
| | Tetrapeptide-7) | | |
| | Crodarom Nut A | 0.01-10.00 | Skin Conditioner |
| | (Water, Propylene | | |
| | Glycol, Walnut | | |
| | Extract) | | |
| | Erythrulose | 0.01-5.00 | Self-Tanning |
| | Sodium Hyaluronate | 0.01-6.00 | Moisterizer |
| | Tocopherol | 0.001-5.00 | Skin Conditioner |
| | Diazolidinyl Urea | 0.01-0.50 | Preservative |
| | Idopropynyl | 0.001-0.50 | Preservative |
| | Butylcarbamate | | |
| | Fragrance | 0.001-1.50 | Fragrance |

⁽¹⁾An abbreviation for “by weight,” used in chemistry and pharmacology to describe the concentration of a substance in a mixture of solution. For example, 30.00 to 85.00 w/w means that the mass of the substance is 30.00 to 85.00 percent of the total mass of the solution or mixture.

From the above, it becomes apparent that the facial sunless or self-tanning composition having anti-wrinkle/anti-aging properties contains from about 30 to 85 weight percent water, from about 0.5 to about 17 weight percent self-tanning agents, from about 0.1 to about 10 weight percent of peptides compatible with the self-tanning agents the peptides functioning as anti-wrinkle/anti-aging agents, from about 0.001 to about 15 weight percent skin conditioners, from about 0.1 to about 6 weight percent moisturizers, from about 1.5 to about 14 weight percent emollients and from about 0.5 to about 4 weight percent of film formers. In addition, the composition further includes minor effective amounts of pH adjusters which provide the composition with a pH in the range of from about 3.25 to about 3.75, opacifiers, one or more colorants to provide the composition with a predetermined color, chelating agents and fragrances to provide the composition with a pleasing aroma. In each of the above ingredients it is important that a safe and effective amount of the ingredients be incorporated into the formulation to achieve the desired effects of such constituents.

Examples of self-tanning agents which may be employed in the practice of the present invention include, but are not limited to, dihydroxyacetone, erythrulose, melanin, mahakanni, walnut extract, combinations thereof and the like.

Anti-wrinkle, anti-aging additives employed in the formulation in the sunless self-tanning composition includes both naturally occurring peptides and commercially available compositions that contain peptides. Further, any peptide compatible with the self-tanning agents of the composition of the present invention may be employed. Examples of such peptides include, but are not limited to, peptide amino acids which are components of collagen and which stimulate fibroblasts, acetyl, hexapeptide-3, palmitoyl oligopeptide, palmitoyl tetrapeptide-7, di-, tri- and tetra-peptides, combinations and derivatives thereof as well as mixtures of such peptides with emollients, humedants, emulsifiers, emulsion stabilizers and/or viscosity control agents. Commercially available compositions which can be employed as an anti-aging/anti-wrinkle additive in the formulation of compositions of the present invention include, but are not limited to, Matrixy™ and Matrixy™ 3000, distributed by Sederma, Inc. of Elison, N.J.; Cellex C™, distributed by Cellex-C International, Inc. of Toronto, Canada; and Physicians Reserve Eye Complex™, Civant Labs of Aurora, Ill.

The term “safe and effective amount” as used herein means that the amount of a compound or composition is sufficient to significantly induce a positive benefit, including independently or in combination the benefits disclosed herein, but low enough to avoid serious side effects, (i.e., to provide a reasonable benefit to risk ratio, within the scope of sound judgment of the skilled artisan).

The term “film former” as used includes polymers for aiding the film forming properties and substantivity of the composition of the present invention.

The term “chelating agent” as used herein is to be understood to mean an active agent capable of removing a metal from a composition by forming a complex so that the metal ion cannot readily participate in or catalyze chemical reactions. The inclusion of a chelating agent is especially useful for providing protection against UV radiation which can contribute to excessive scaling or skin texture agents or against other environmental agents which can cause skin damage.

The term “skin conditioners” as used herein include humectants, moisturizers, skin conditioners, and the like. These materials include, but are not limited to, guanidine; urea; glycolic acid and glycolate salts (e.g., ammonium and quaternary alkyl ammonium); salicylic acid; lactic acid and lactate salts (e.g. ammonium and quaternary alkyl ammonium); aloe vera in any of its variety of forms (e.g. aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g. alkoxylated glucose, fructose, gucosamine); hyaluronic acid, lactamide monoethanolamine; acetamide monoethanolamine; panthenol; allantoin; and mixtures thereof.

The term “viscosity controlling agents,” “emulsifying agents,” and “emulsion stabilizer(s),” as used herein function as thickening and gelling agents and include, but are not limited to, gums, such as, acacia, agar, algin, alginic acid, ammonium alignate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Preferred compositions of the present invention include a thickening agent selected from carboxylic acid polymers, crosslinked polyacrylate polymers, polyacrylamide polymers, and mixtures thereof, more preferably selected from carboxylic acid polymers, polyacrylamide polymers and mixtures thereof.

The term “emollient” as used herein refers to a material useful for the prevention or relief of dryness, as well as for the protection of the skin. A wide variety of suitable emollients are known and may be used herein.

In formulating the compositions of the present invention the constituents of phase A of the formulation are blended together at a temperature of from about 70° C. to about 80° C. Once a uniform mixture has been formed of the constituents of phase A the ingredients of phase B are admixed while heating at a temperature of from about 70° C. to about 80° C. When a substantially uniform mixture of the constituents of phase B has been achieved, the resulting mixture of the phase B constituents is admixed with the mixture formed from the constituents of phase A. The admixing of the constituents of phase A and B is carried out at a temperature of from about 70° C. to about 80° C. and the resulting mixture is constantly stirred while forming the admixture. In addition, the mixture is stirred for a period of time of about 10 minutes at a temperature of from about 70° C. to about 80° C. Thereafter, the resulting mixture is allowed to cool to a temperature of from about 60° C. to about 70° C. at which time the constituents of phase C are admixed into the resulting mixture formed by admixing the constituents of phase A and phase B. Once a substantially uniform mixture is formed from phases A, B, and C, each constituent of phase D is added individually to the mixture, ( i.e., the constituents of phase D are not premixed). In addition, the ingredients of phase D are introduced into the resulting mixture of phases A, B, and C at a temperature in the range of from about 50° C. to about 60° C. Once a substantially uniform mixture has occurred, the constituents of phase E are admixed separately to form a uniform mixture. Once a uniform mixture is formed from the constituents of phase E, the phase E mixture is introduced into the mixture containing the constituents of phases A, B, C and D. The admixing of the constituents of phase E with the mixture formed from admixing the constituents of phases A, B, C and D is carried out at a temperature of from about 40° C. to about 50° C. Lastly, the constituents of phase F are added to the mixture produced from admixing the constituents of phases A, B, C, D and E and the resulting mixture is mixed at room temperature for a period of time effective to provide a uniform blend or mixture of the composition of the present invention.

It should be noted that by admixing the phases as described above, a substantially uniform stable product is achieved which can be employed as a facial self-tanning composition having anti-wrinkle and anti-aging properties.

In order to more fully describe the present invention the following example is set forth. However, it is to be understood that the example is for illustrative purposes only and is not to be construed as limiting the invention disclosed herein.

### EXAMPLE

| | W/W | | |
|---|---|---|---|
| Phase | Percent | Raw Material | INCL |
| A | 63.9850 | DI Water | Purified Water |
| A | 0.1000 | Xanthan Gum, FCC | Xanthan Gum |
| A | 0.0500 | D&C Green No. 5 (DA- | Green 5 |
| | | 3805) | |
| A | 0.0550 | FD&C Red No. 40 (DA- | Red 40 |
| | | 6056) | |
| A | 0.0300 | FD&C Yellow No. 5 (DA- | Yellow 5 |
| | | 4400) | |
| A | 0.2000 | Tetrasodium EDTA | Tetrasodium EDTA |
| A | 0.2500 | Methylparaben | Methylparaben |
| B | 3.0000 | Jojoba Oil | Simmondsia |
| | | | Chinensis (Jojoba) |
| | | | Seed Oil |
| B | 2.5000 | Dermol 818 | Ethylhexyl |
| | | | Stearate |
| B | 2.0000 | Dimethicone 47 V10 | Dimethicone |
| B | 0.5000 | Liponate SPS | Cetyl Ester |
| B | 1.0000 | Cyclomethicone | Cyclopentasiloxane |
| B | 1.0000 | Cosmowax J | Cetearyl Alcohol, |
| | | | Ceteareth-20 |
| B | 2.0000 | Lexorez TL-8 | Trimethylpentanediol/ |
| | | | Adipic Acid |
| | | | Copolymer |
| B | 1.5000 | Lipomulse 165 | Glyceryl Stearate, |
| | | | PEG-100 Stearate |
| B | 2.5000 | Lipo GMS 450 | Glyceryl Stearate |
| B | 0.1500 | Propylparaben | Propylparaben |
| C | 2.0000 | Sepigel 305 | Polyacrylamide, |
| | | | C13-14 |
| | | | Isoparaffin, |
| | | | Laureth-7 |
| D | 1.0000 | Lactic Acid | Lactic Acid |
| D | 0.4300 | Triethanolamine | Triethanolamine |
| E | 5.0000 | DI Water | Purified Water |
| E | 3.0000 | Trivalin SF | Ethoxydiglycol |
| E | 4.0000 | Dihydroxyacetone (DHA) | Dihydroxyacetone |
| F | 3.0000 | Matrixyl 3000 | unknown |
| F | 3.0000 | Crodarom Nut A | Purified Water, |
| | | | Proylen Glycol, |
| | | | Juglans Regia |
| | | | (Walnut) Extract |
| F | 0.2500 | Erythrulose | Erythrulose |
| F | 0.1000 | Sodium Hyaluraonte | Sodium |
| | | | Hyaluronate |
| F | 0.1000 | Tocopherol | Tocopherol |
| F | 0.2000 | Germall Plus | Diazolidinayl Urea |
| | | | Iodopropynyl |
| | | | Butylcarbmate |
| F | 0.1000 | Ocean Fragrance #287-241 | Fragrance |

In formulating the compositions of the present invention the constituents of phase A are blended together at a temperature of about 75° C. Once a uniform mixture has been formed of the constituents of phase A the ingredients of phase B are admixed while heating at a temperature of about 75° C. Once a substantially uniform mixture of the constituents of phase B has been achieved, the resulting mixture is admixed with the mixture formed from the constituents of phase A. The admixing of the constituents of phase A and B is carried out at a temperature of from about 75° C. and the resulting mixture is constantly stirred while forming the admixture. In addition, the mixture is stirred for a period of time of about 10 minutes at a temperature of from about 75° C. Thereafter, the resulting mixture is allowed to cool to a temperature of from about 65° C. at which time the constituents of phase C are admixed into the resulting mixture formed by admixing the constituents of phase A and phase B. Once a substantially uniform mixture is formed by admixing the constituents of phases A, B, and C, each constituent of phase D is added individually to the mixture, ( i.e., the constituents of phase D are not premixed). In addition, the constituents of phase D are introduced into the resulting mixture of the constituents of phases A, B, and C at a temperature in the range of from about 55° C. Once a substantially uniform mixture has occurred, the constituents of phase E are admixed separately to form a uniform mixture. Once a uniform mixture is formed from the constituents of phase E, the phase E mixture is introduced into the mixture containing the constituents of phases A, B, C and D. The admixing of the constituents of phase E with the mixture formed from admixing phases A, B, C and D is carried out at a temperature of from about 45° C. Lastly, the constituents of phase F are admixed individually and at room temperature to the mixture produced from the admixing of the constituents of phases A, B, C, D and E. Thereafter, the resulting mixture is admixed at room temperature for a period of time effective to provide a uniform blend or mixture.

It should be noted that by admixing the constituents of phases as described above, a substantially uniform stable product is achieved which can be employed as a facial self-tanning composition having anti-wrinkle and anti-aging properties.

What is claimed is:

1. A facial sunless and self-tanning composition having a pH in the range of from about 3.25 to about 3.75, and anti-wrinkle and anti-aging properties, the composition comprising: an emulsified oil phase/water phase wherein, the water phase of the emulsified oil phase/water phase comprises a major amounts of water and safe and effective minor amounts of viscosity modifiers, colorants, at least one chelating agent and preservatives and wherein, the oil phase of the emulsified oil phase/water phase comprises safe and effective amounts of at least one sunless/self-tanning agent, at least one anti-wrinkle/anti-aging agent which is compatible with the at least one sunless/self-tanning agent, skin conditioners, emollients, viscosity modifiers, opacifiers, moisturizers, preservatives and fragrances.

2. The facial sunless and self-tanning composition of claim 1 wherein the amount of water present in the water phase is from about 30 to about 85 weight percent, based on the total weight of the composition, the safe and effective minor amounts of viscosity modifiers present in the water phase is from about 0.1 to about 0.5 weight percent, based on the total weight of the composition, the safe and effective minor amounts of colorants present in the water phase is from about 0.003 to about 0.9 weight percent, based on the total weight of the composition, the safe and effective minor amounts of the at least one chelating agent present in the water phase is from about 0.01 to about 0.5 weight percent, based on the total weight of the composition, and the safe and effective minor amounts of preservatives is from about 0.05 to about 0.5 weight percent, based on the total weight of the composition.

3. The facial sunless and self-tanning composition of claim 2 wherein the at least one sunless/self-tanning agent is present in the oil phase in an amount of from about 0.5 to about 17 weight percent, based on the total weight of the composition, the at least one anti-wrinkle/anti-aging agent is present in the oil phase in an amount of from about 0.1 to about 10 weight percent, based on the total weight of the composition, the skin conditioners are present in the oil phase in an amount of from about 0.01 to about 15 weight percent, based on the total weight of the composition, the moisturizers are present in the oil phase in an amount of from about 0.1 to about 6 weight percent, based on the total weight of the composition, the emollients are present in the oil phase in an amount of from about 1.5 to about 14 weight percent, based on the total weight of the composition, the film formers are present in the oil phase in an amount of from about 0.5 to about 4 weight percent, based on the total weight of the composition, the opacifiers are present in the oil phase in an amount of from about 0.25 to about 3 weight percent based on the total weight of the composition and the fragrances are present in the oil phase in an amount of from about 0.001 to about 1.5 wieght percent based on the total weight of the composition and safe and effective minor safe amounts of opacifiers and fragrances.

4. The facial sunless and self-tanning composition of claim 3 wherein the at least one sunless/self-tanning agent is selected from the group consisting of dihydroxyacetone, erythrulose, melanim, mahakanni, walnut extract and combinations thereof.

5. The facial sunless and self-tanning composition of claim 4 wherein the at least one anti-wrinkle/anti-aging agent is a peptide.

6. The facial sunless and self-tanning composition of claim 4 wherein the at least one anti-wrinkle/anti-aging agent is selected from the group consisting of acetyl hexapeptide-3, peptide amino acids, palmitoyl oligopeptide, palmitoyl tetrapeptide-7, di-, tri- and tetra-peptides, and combinations and derivatives thereof and a mixture of glycerine, butylene, glycol, carbon, polysorbate-20, palmitoyl oligopeptide, palmitoyl tetrapeptide-7.

7. The facial sunless and self-tanning composition of claim 1 wherein the at least one sunless/self-tanning agent is selected from the group consisting of dihydroxyacetone, erythrulose, melanim, mahakanni, walnut extract and combinations thereof.

8. The facial sunless and self-tanning composition of claim 1 wherein the at least one anti-wrinkle/anti-aging agent is a peptide.

9. The facial sunless and self-tanning composition of claim 8 wherein the at least one anti-wrinkle/anti-aging agent is selected from the group consisting of acetyl hexapeptide-3, peptide amino acids, palmitoyl oligopeptide, palmitoyl tetrapeptide-7, di-, tri- and tetra-peptides, and combinations and derivatives thereof and a mixture of glycerine, butylene, glycol, carbon, polysorbate-20, palmitoyl oligopeptide, palmitoyl tetrapeptide-7, and.

10. A method for producing a facial sunless and self-tanning composition having anti-wrinkle/anti-aging properties, the method comprising the steps of:
admixing water and safe and effective minor amounts of viscosity modifiers, colorants, at least one chelating agent and preservatives at a temperature of from about 70° C. to about 80° C. for a period of time effective to form a substantially uniform phase A aqueous mixture;
mixing effective amounts of emollients, at least one opacifier, and an emulsifier at a temperature of from about 70° C. to about 80° C. for a period of time to provide a substantially uniform phase B mixture;
admixing the phase A aqueous mixture with the phase B mixture at a temperature of from about 70° C. to about 80° C. for a period of time effective to provide a substantially uniform phase B mixture;
permitting the resulting mixture formed by admixing phases A and B to cool to a temperature of from about 60° C. to about 70° C.;
admixing effective amounts of a film former, emulsifier and a preservative to form a substantially uniform phase C mixture;
admixing the phase C mixture with the resulting mixture containing phases A and B at a temperature of from about 60° C. to about 70° C. from a period of time to form a substantially uniform mixture of phases A, B, and C;
cooling the substantially uniform mixture of phases A, B and C to a temperature of from about 55° C. to about 60° C.;
admixing pH adjusters constituting phase D with the substantially uniform mixture of phases A, B and C at a temperature of from about 50° C. to about 60° C. to form a substantially uniform mixture of phases A, B, C and D;
admixing effective amounts of water, a solubilizer and a sunless/self-tanning agent to form a substantially uniform phase E mixture;
admixing the phase E mixture with the mixture containing phases A, B, C and D at a temperature of from about 40° C. to about 50° C. for a period of time to form a substantially uniform mixture containing the constituents of phases A, B, C and D;
permitting the mixture containing the mixture of phases A, B, C, D and E to cool to room temperature;
admixing a peptide having anti-wrinkle/anti-aging properties, skin conditioners, and additional self-tanning agents, moisturizers, preservatives and fragrances at room temperature to provide a substantially uniform phase F mixture; and
admixing the phase F mixture with the phase A, B, C, D and E mixtures at room temperature to provide a facial sunless/tanning composition containing anti-wrinkle and anti-aging properties, the facial sunless/tanning composition having a pH in the range of from about 3.25 to about 3.75, anti-wrinkle and anti-aging properties.

11. The method of claim 10 wherein:
the phase A aqueous mixture contains from about 30 to about 85% w/w water, from about 0.01-0.50% w/w of a viscosity modifier, from about 0.0003-0.8% w/w colorants, from about 0.0.01-0.5% w/w of a chelating agent and from about 0.05-0.5% w/w of a preservative;
the phase B mixture contains from about 1.6-19 emollients, from about 0.10-4.00% w/w thickeners, from about 0.25-3.00% w/w opacifiers and from about 0.25-3.00% w/w emulsifiers;
the phase C mixture contains from about 0.25-5.00% w/w emulsifiers;
the phase D mixture contains from about 0.11-9.00% w/w pH adjusters;
the phase E mixture contains from about 2.0-8.00% w/w deionized water base, from about 0.10-8.00% w/w solubilizer and from about 0.50-12.00% w/w self tanning agent;
the phase F mixture contains from about 0.01-10.00% w/w peptides, from about 0.011-15.00% w/w skin conditioners, from about 0.01-6.00% w/w moisturizers, from about 0.011-1.0% w/w preservatives and from about 0.001-1.50% w/w fragrances.

12. The method of claim 11 wherein the viscosity modifier is xanthan gum, the colorants are selected from the group consisting of D & C Green No. 5, FD & C Red No. 40, FD & C Yellow No. and combinations thereof, the chelating agent is tetrasodium EDTA and the preservative is methylparaben.

13. The method of claim 11 wherein the emollients are selected from the group consisting of simmondisia chinensis (jojoba) oil, ethylhexyl stearate, dimethicone, and cyclophentasiloxane, the thickener is cetyl esters, the opacifier is cetearyl alcohol, the emulsifiers are selected from a group consisting of ceteareth-20, PEG-100 stearate and glyceryl stearate, the film former is trimethylpentan-ediol/adipic acid copolymer and the preservative is propylparaben.

14. The method of claim 11 wherein the emulsifier is polyacrylamide, C13-14 isoparaffin, laureth-7.

15. The method of claim 11 wherein the pH adjusters are selected from a group consisting of lactic acid and triethanolamine.

16. The method of claim 11 wherein the base is deionized water, the solubilizer is ethoxydiglycol, and the self-tanning agent is dihydroxyacetone.

17. The method of claim 11 wherein the peptide is a mixture of glycerin, butylene glycol, carbomer, polysorbate-20, palmitoyl oligopeptide, palmitoyl tetrapeptide-7, the skin conditioners are selected from a group consisting of a mixture of water, propylene glycol, walnut extract and tocopherol, the self-tanning agent is erythrulose, the moisturizer is sodium hyaluronate, the preserveratives are selected from a group consisting of diazolidinyl urea and idopropynyl butylcarbamate.
